(19) United States
(12) Patent Application Publication
(10) Pub. No.: US 2019/0003831 A1
(43) Pub. Date: Jan. 3, 2019

(54) DEVICE FOR DETECTING DEFORMATION OF A HOLLOW COMPONENT

(71) Applicant: DEPUY IRELAND UNLIMITED COMPANY, RINGASKIDDY COUNTY CORK (IE)

(72) Inventors: ANDREW BAILEY, LEEDS (GB); STEPHANIE PRINCE, WAKEFIELD (GB); DUNCAN YOUNG, MELBOURN (GB)

(21) Appl. No.: 16/064,482

(22) PCT Filed: Dec. 19, 2016
(86) PCT No.: PCT/EP2016/081709

§ 371 Date: Jun. 21, 2018

(30) Foreign Application Priority Data
Dec. 23, 2015 (GB) 1522723.4

(51) Int. Cl. G01B 21/32; G01B 21/22; A61F 2/46; A61F 2/34

(52) U.S. Cl. CPC G01B 21/32; A61F 2/34; A61F 2/4609; G01B 21/22

(57) ABSTRACT

The invention provides a device for fitting into a hollow component to provide an indication of whether the hollow component has been deformed, which includes a frame which can be fitted in the) component, and an elongate indicator suspendedly connected to the frame, with any deformation of the frame being visualised as a deflection of the indicator.

### FIELD OF THE INVENTION

The present invention relates to a device for detecting the deformation of a hollow component and methods of using the device. In particular, the present invention concerns a device for detecting the deformation of a hollow orthopaedic component, such as a cup component of an orthopaedic joint prosthesis.

### BACKGROUND TO THE INVENTION

Press-fit fixation is a common technique for implanting components of an orthopaedic prosthesis that eliminates the need for supplementary fixation such as cement, screws or spikes. Short term stability of the implant is achieved through an interference fit between the implant and the surrounding bone. Long term stability is achieved through bone ingrowth or on growth, which is typically aided by an exterior roughened or porous surface. Such fixation techniques are applicable to fixation of cup components of orthopaedic joint prostheses. They are applicable for example to fixation of an acetabular component of a hip joint prosthesis. They are applicable to fixation of the glenoid component of an anatomic shoulder joint prosthesis. They are applicable to fixation of the humeral component of a reverse shoulder joint prosthesis.

An interference fit results from the component being generally larger than the prepared implantation site. For instance, during hip arthroplasty a hemi-spherical porous-coated acetabular component that is typically 1 to 4 mm larger than the last reamer used to prepare the patient's acetabulum will be forcefully impacted into the acetabulum. An interference fit is formed between the acetabular component and the host-bone.

Squire et al (J Arthroplasty 2006 Sep; 21(6 Suppl 2):72-7) demonstrated that the compressive forces acting on the acetabular component during this forceful insertion can cause deformation of the component, as evidenced by a change in the geometry of the rim of the acetabular component from circular, for example towards elliptical. This deformation has negative clinical consequences, including those resulting from alterations in the bearing geometry and the inability of liners to be correctly aligned and seated.

It is therefore of clinical importance that a surgeon can pre- and/or intra-operatively detect whether an acetabular component is deformed. A surgeon can then decide if the acetabular cup is too deformed to be used. A surgeon might use information concerning the extent of deformation of the acetabular cup component to assess whether additional reaming of the acetabulum is required. It might be that the extent of the deformation is insignificant and will not have negative clinical consequences, or that the deformation will resolve following implantation. However, the amount of deformation is small and can be difficult to judge by eye, particularly in the case of thin-walled acetabular components.

Squire et al (in the paper identified above) have proposed that measurements of the deformation of an acetabular component can be made using a telescoping gauge in combination with a measuring instrument such as a pair of Vernier callipers. In this two-step technique, a gauge is inserted into the acetabular component, locked in place, and then the distance between the locked gauge ends measured using Vernier callipers. This is a time-consuming technique. It requires the user to have a familiarity with reading a Vernier calliper. It also requires dexterous handling of the instruments using both hands. There is also a need for additional sterile instrumentation within the inventory.

### SUMMARY OF THE INVENTION

The invention provides a device for fitting into a hollow component to provide an indication of whether the hollow component has been deformed, which includes a frame which can be fitted in the component, and an elongate indicator suspendedly connected to the frame, with any deformation of the frame being visualised as a deflection of the indicator.

Accordingly, the invention provides a device for fitting into a hollow component to provide an indication of deformation of the hollow component, the device comprising:
a frame, and
an elongate indicator suspendedly connected to the frame, the elongate indicator being arranged to deflect as a result of deformation of the frame.

The device provided by the invention can provide a user with a readily discernible indication that the hollow component with which it is used has been deformed, through angular deflection of the indicator from the position which it adopts in the absence of deformation. The device can be positioned in the hollow component before the component is exposed to a force which might cause it to be deformed so that deformation can then be detected during application of a deforming force. The device can be positioned in a hollow component to determine whether or not it has already been deformed. It is possible to construct the device so that readily discernible deflection of the indicator follows only small deformations of the frame (for example of the order of less than 3% of the transverse dimension of the hollow component (which will be its diameter when the component is circular when viewed in cross-section), or less than 2.5%, or less than 2%, or less than 1%).

The elongate indicator can be suspendedly connected to the frame by a pair of arms. A first arm extends inwardly from a first point on a first side of the frame, and a second arm extends inwardly from a second point on a second side of the frame, the second side being opposite to the first side. The elongate indicator has a longitudinal axis to which the first and second arms are connected at spaced apart points along the axis to suspendedly connect the elongate indicator to the frame. A change in the distance between the first and second points on opposite sides of the frame caused by deformation of the frame results in angular deflection of the indicator.

The angular deflection response of the indicator to deformation of the frame is affected by the distance between the arms. Frequently, the length of the first arm will be equal to the length of the second arm. This can help to provide a controlled angular deflection of the indicator in response to deformation of the frame. Optionally, when the first and second arms have the same length, the ratio of the length of the arms to the distance between the arms measured along the indicator is at least about 0.3, or at least about 0.5, or at least about 1, or at least about 1.5, or at least about 2, or at least about 2.5. Optionally, the value of the ratio is not more than about 6, or not more than about 5, or not more than about 4.5, or not more than about 4, or not more than about 3.5.

Optionally, the first arm is approximately parallel to the second arm when there is no deformation of the frame. It will often be the case that arms which are parallel in a device prior to any deformation will remain parallel when the device (and a hollow component in which the device is located) is deformed, dependent on the nature of the deformation of the device.

The angle between each of the first and second arms and the indicator may be approximately 90° when there is no deformation of the frame. Angles other than approximately 90° are envisaged between each of the first and second arms and the indicator prior to any deformation. For example, the angle between one or each of the arms, and the portion of the indicator which extends between the arms, can be at least about 60°, or at least about 70°, or at least about 80°. The said angle can be not more than about 120°, or not more than about 110°, or not more than about 100°.

Hinges can allow the angle between each of the first and second arms and the indicator to change when the device is deformed so that the angle between each of the first and second arms and the indicator may be varied. A hinge may be provided towards the end of the arm at which the arm is connected to the indicator. Advantageously, the hinge is provided at the end of the arm at which the arm is connected to the indicator. This results in a greater range of motion of the indicator, and therefore a greater ability to magnify the deformation of the frame. In other arrangements, a hinge might be provided between the opposite ends of the arm. The connection between the arm and the indicator might then be relatively rigid.

The hinge may be provided by a portion of the arm that is thinner compared to the rest of the arm. Such a hinge arrangement is sometimes referred to as a living hinge. The use of such hinges can enable the two arms and the indicator to be formed as one piece, for example by moulding. The thinness of the arm is such that the variation in the angle between the arm and the indicator caused by the arm being subjected to a force takes place mainly in the thinner portion rather than in the remainder of the arm. This provides a more accurate response and articulation of the indicator.

It can be preferred that the wall of the arm in the region of a thinned hinge portion is rounded when the arm is viewed from one side so that there is no sharp change in wall thickness at the point at which the hinge will flex when the device is deformed. This can help to reduce stress concentrations at the hinge which could result in the weakening and possible failure of the hinge.

The device can also include a second elongate indicator that is suspendedly connected to the frame. This second elongate indicator allows deformation of the hollow component along two axes to be detected.

The second elongate indicator can be suspendedly connected to the frame by a second pair of arms. The second pair of arms includes a third arm that extends inwardly from a third point on a third side of the frame, and a fourth arm that extends inwardly from a fourth point on a fourth side of the frame, the fourth side being opposite to the third side. The second elongate indicator has a longitudinal axis to which the third and fourth arms are connected at spaced apart points along the axis.

Optionally, the frame has a continuous periphery. Deformation of a device in which the frame is continuous around the periphery of the device, as a result of application of a compressive force to a hollow component in which the device is positioned, will result in a reduction of a first dimension of the device and an increase of a second dimension of the device, where the first dimension is measured transverse to the second dimension. A device in which the frame is continuous around the periphery of the device can be used to detect deformation of a hollow component in a direction which is generally aligned with one or both of the arms in the device, as well as in directions which are not aligned with one or both of the arms, for example in a direction which is generally perpendicular to one or both of the arms or a direction which forms an acute angle with one or each of the arms.

The frame should be configured so that it is a snug fit in the hollow component in which it is intended that the device should be used. When the frame extends continuously around the periphery of the device, it will generally be preferred that the shape of the frame should complement the internal shape of the hollow component so that the frame is in contact with the component around the periphery of the device.

A frame which does not extend continuously around the periphery of the device can have spaced apart frame portions around the periphery of the device. The frame portions will generally be arranged so that they can contact the hollow component at points which are on or close to the axis along which compressive forces will be applied to the component.

Frequently, the hollow component will be circular and deformation of the component which is to be detected using the device of the invention will involve deformation from circular, for example towards a generally oval or elliptical shape. This shape change can be detected by an angular deflection of the indicator.

Indicia can be provided on the device to help a user to recognise or to quantify the angular deflection of the indicator. Indicia can be provided on the frame, especially in the portion of the frame which is adjacent to an end of the indicator prior to any deformation. Frequently, the indicator will have first and second opposite ends. Indicia can then be provided adjacent to each end of the indicator. Indicia can be provided on the frame when the frame is moulded if a moulding technique is used to make the frame. Indicia can be marked on the device using a material or technique which leads to the indicia being appropriately contrasting relative to the surface on which they are provided. Indicia might be provided by laser marking in some circumstances.

The indicia may enable the user to quantify the amount of deflection of the indicator. The indicia may be provided as a graduated scale. Indicia may be provided that only indicate to the user that a certain amount of deformation of the hollow component been attained or exceeded. This amount may be a clinically relevant amount of deformation of an orthopaedic cup component. The indicia may include a distinctive marking, especially a marking in a contrasting colour, for example as a red line. Deflection of the indicator to or beyond this line visually indicates to the user that a certain amount of deformation of the hollow component has been attained or exceeded.

Optionally, the device further includes a marker rotatably mounted on the frame and arranged for deflection by the elongate indicator to thereby mark a largest angular deflection of the elongate indicator from a series of angular deflections of the elongate indicator when the device is placed within the hollow component in two or more orientations.

The marker is arranged to be angularly deflected from a first position to a second position in response to a first angular deflection of the elongate indicator and not automatically returned to the first position. The marker is also arranged to be angularly deflected from the second position to a third position in response to a second angular deflection of the elongate indicator when the second angular deflection is greater than the first angular deflection. If the second angular deflection is less than or equal to the first angular deflection, the marker will remain in the second position.

The fact that the marker remains in the third position and does not automatically revert to its original position enables a user to orientate the device in two or more orientations within the hollow component, for example by rotating the device within the hollow component, thereby to determine the largest amount of angular deflection (indicated by the third position) of the elongate indicator. The largest amount of angular deflection detected by the marker indicates the largest amount of deformation of the hollow component. This can help the user to identify if the hollow component has been deformed beyond a predetermined acceptable limit. If this is the case the hollow component might need to be discarded. In the case of a trial acetabular shell, this can also provide the surgeon with information as to whether the patient's acetabulum requires additional reaming before the final acetabular shell implant is implanted, and the liner is inserted.

In some constructions, the elongate indicator and the marker are each rotatably mounted for rotation about a common rotation axis.

The marker may be connected to a plate that extends between the first and second points on the frame. The plate may be removably connectable to the frame.

Advantageously, at least one indicium is provided on the plate for indicating the amount of deflection of the second deflection indicator. This indicium may be in the form of a single line that which represents the acceptable tolerance of the hollow component to deformation. If the marker deflects past this point, then the user is aware that the deformation of the hollow component is outside of acceptable limits. The user can then decide whether the hollow component should be discarded, or whether the site into which the hollow component is to be fitted/implanted requires further preparation.

The frame and elongate indicator of device may be made from the same material, for example a flexible polymer. The first and/or second pair of arms may be made from the same material as the frame and elongate indicator. Each individual component (frame, arms and indicator) can be designed so that it has an appropriate flexibility relative to the other components to ensure that the frame will deform with the hollow component, and the arms will then act appropriately on the indicator to deliver a controlled angular deflection of the indicator. When the device is intended for use within medical applications, the material should be capable of withstanding the conditions to which it will be exposed during a sterilisation procedure, for example involving exposure to elevated temperature or to radiation. Examples of suitable polymer materials include polyesters, polyamides and polyolefins, including polyphenylsulfone Radel® PPSU sold by Solvay, and the acetal copolymer Celcon® sold by Ticona.

It can be preferred for many applications to provide the frame and elongate indicator of the device as one piece. It may also be preferred for many applications to provide the frame, elongate indicator and at least one of the pairs of arms as one piece. This might be achieved by manufacturing at least these parts of a device by a moulding technique, for example by injection moulding. The device may be intended to be disposed after a single use.

In some embodiments the indicator is a needle and the first and second arms are connected to the needle at spaced apart points along the length of the needle.

The device can be used to detect deformation of a component of a medical device, especially a component which is to be used in an orthopaedic surgical procedure which could be an implant component or trial implant component for use as an instrument. The device can be used to detect deformation of a hollow component of an orthopaedic joint prosthesis which is intended to engage a concave head component of a corresponding component to enable articulation between the head component and the hollow component. It can also be used to detect deformation of a trial hollow component which is used in an orthopaedic surgical procedure. Examples of hollow components with which the device of the invention can be used include components (trial and implant components) for placing in a cavity in a patient's glenoid in a surgical procedure to implant an anatomic shoulder joint prosthesis, components (trial and implant components) for placing in a cavity in a patient's humerus in a surgical procedure to implant a reverse shoulder joint prosthesis. The device of the invention is particularly well suited for use with components (trial and implant components) for placing in a cavity in a patient's acetabulum in a surgical procedure to implant a hip joint prosthesis.

Optionally, in devices that are to be used in detecting deformation of components (trial and implant components) during placement in a cavity in a patient's acetabulum the device is provided with an inner frame having an internal periphery that is dimensioned to receive an acetabular cup insertion instrument. For example, the internal periphery can be dimensioned to receive the shaft of the instrument. Impingement of the device causes the inner frame to rotate around the shaft of the instrument, which in turn causes angular deformation of the indicator.

The inner frame can be provided as a component of one of the first or second arms of the device. For example, the first arm or the second arm may include a first portion and a second portion, with the first portion being separated from the second portion by an inner frame.

The inner frame can be provided as a component of the indicator. For example, the indicator can comprise a first needle portion and a second needle portion, with the first needle portion and the second needle portion extending outwardly from opposite points of the inner frame.

The invention also provides a kit comprising the device of the invention and a hollow component, in which the hollow component has a rim and the device fits snugly within the rim of the hollow component, with the frame in contact with the rim.

The device may be preassembled within the hollow component. This ensures that the correct device is being used in conjunction with the hollow component. It can eliminate need to fit the device inside the hollow component prior to deployment of the hollow component.

In particular, the kit may include the device of the invention and an orthopaedic component. The component can be an implantable component of a joint prosthesis, for example, an acetabular cup component of a hip prosthesis, a glenoid component of an anatomic shoulder joint prosthesis or a humeral component of a reverse shoulder joint prosthesis. The orthopaedic component may be a trial component. The use of the device with a trial component enables a surgeon to ensure that the trial component is appropriately seated within the bone prior to implantation of the component of the prosthesis which is to be implanted in the patient. This prevents unnecessary damage to the final prosthesis. Deflection of the indicator of the device when fitted within a trial component highlights to the surgeon that the trial is either incorrectly or non-optimally seated. The surgeon can then judge whether additional reaming of the surgical site is required.

The kit may include a selection of differently sized hollow orthopaedic components (trial or implant components), with each hollow component having a corresponding device for indicating deformation of the component pre-assembled within it. The kit may include a selection of sizes of hollow components (trial or implant components) such as acetabular cup components, or glenoid cup components, or humeral cup components, each hollow component having a device for indicating deformation of the component pre-assembled within it.

The kit may include a selection of differently sized hollow orthopaedic components (trial or implant components) and a selection of complementary devices for indicating deformation of the hollow component. The user selects and fits the appropriate device into the hollow component.

Optionally, a kit may include a first device and a second device, each device having a frame with a discontinuous periphery, with the lengths of the first and second arms of the first device being different from the lengths of the first and second arms of the second device. The first and second device can be used to measure deformation in differently sized hollow components.

The invention also provides a method of detecting deformation of a hollow component, the method comprising the step of detecting angular deflection of an elongate indicator within a device fitted within the hollow component, the device comprising:
a flexible frame, and
an elongate indicator suspendedly connected to the frame, the elongate indicator being arranged to deflect as a result of deformation of the frame.

The method may be used as part of a quality control procedure. For example, the method may be used by a manufacturer to detect if a hollow component has been deformed during the manufacturing process. The method may also be used to detect whether deformation has occurred during the transportation and/or storage of the hollow component. The device may be pre-assembled within the hollow component.

The method may be used to detect if deformation of a hollow component has occurred during insertion of the hollow component into a cavity. This is of particular use to an orthopaedic surgeon because the deformation of a cup component of an orthopaedic joint prosthesis can have serious clinical consequences if it were to remain undetected. An orthopaedic surgeon may therefore use the method to detect if deformation of a cup component has occurred during implantation. The device may be used to detect deformation of the acetabular component of a hip joint prosthesis, the glenoid component of an anatomic shoulder joint prosthesis or the humeral component of a reverse shoulder joint prosthesis. An orthopaedic surgeon may also use the method during the trialling of a joint prosthesis. In such a method, the surgeon will be able to visualise any deformation of the trial by virtue of the deflection of the indicator, and then make a judgement on whether further preparation of the surgical site, for example by re-reaming, is necessary.

Optionally, the device further includes a marker rotatably mounted on the frame and arranged for deflection by the elongate indicator to thereby mark a largest angular deflection of the elongate indicator from a series of angular deflections of the elongate indicator when the device is placed within the hollow component in two or more orientations and in which the method further comprises the step of detecting the largest angular deflection of the elongate indicator when the device is placed within the hollow component in two or more orientations.

### DETAILED DESCRIPTION OF THE INVENTION

The invention will now be described, by way of example only, with reference to the following drawings, in which:

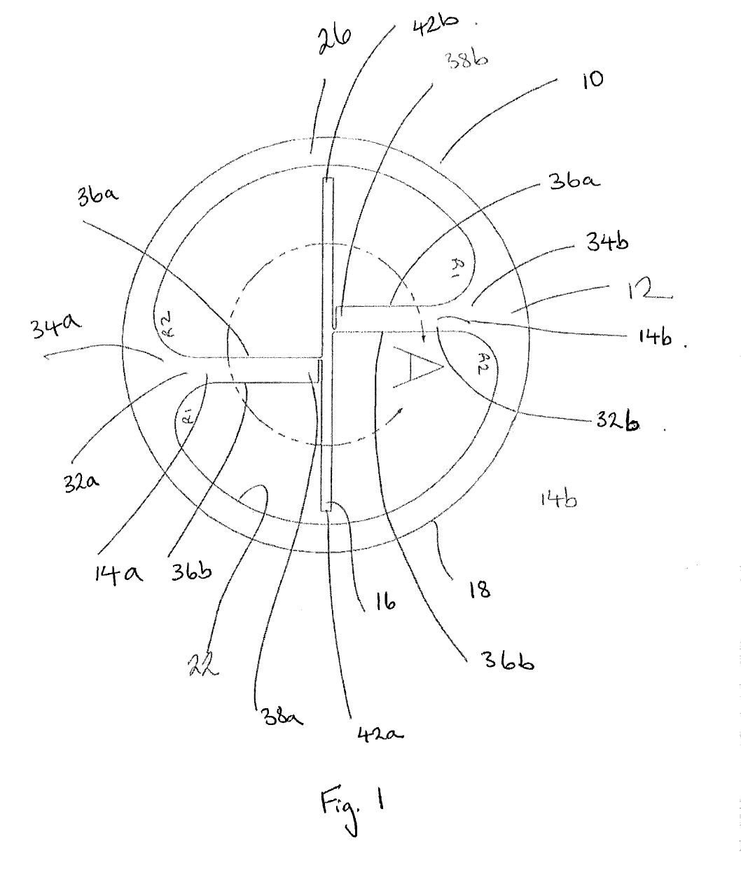

FIG. 1 is a top elevation view of a first construction of a device for detecting deformation of a hollow component in an un-deformed state.

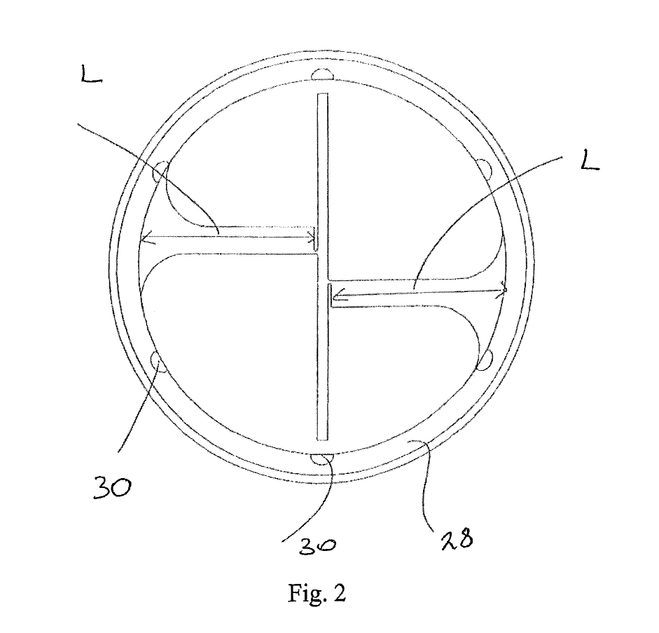

FIG. 2 is a bottom elevation view of the device of FIG. 1.

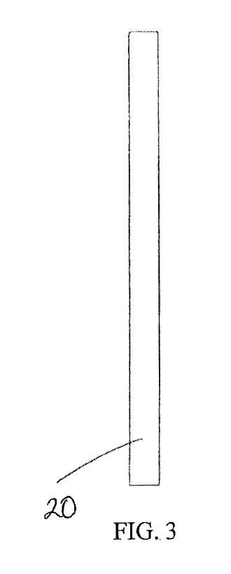

FIG. 3 is a side elevation view of the device of FIG. 1.

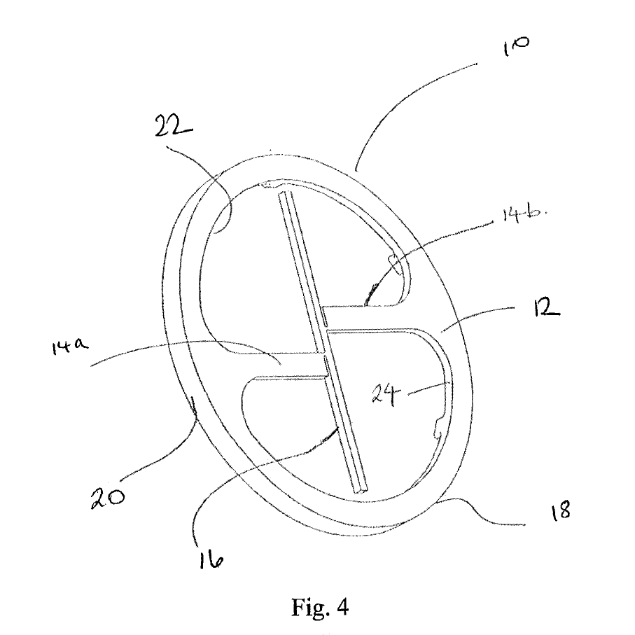

FIG. 4 is a perspective view of the device of FIG. 1

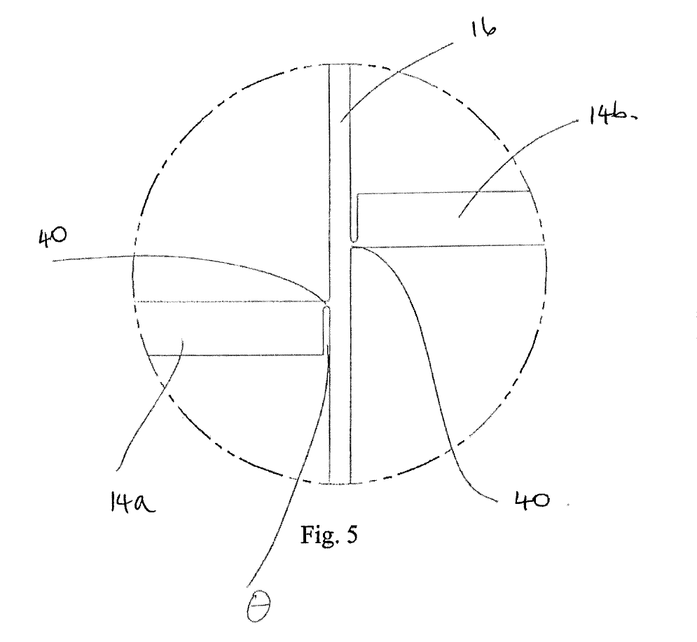

FIG. 5 is an enlarged view of the area “A” of the device of FIG. 1.

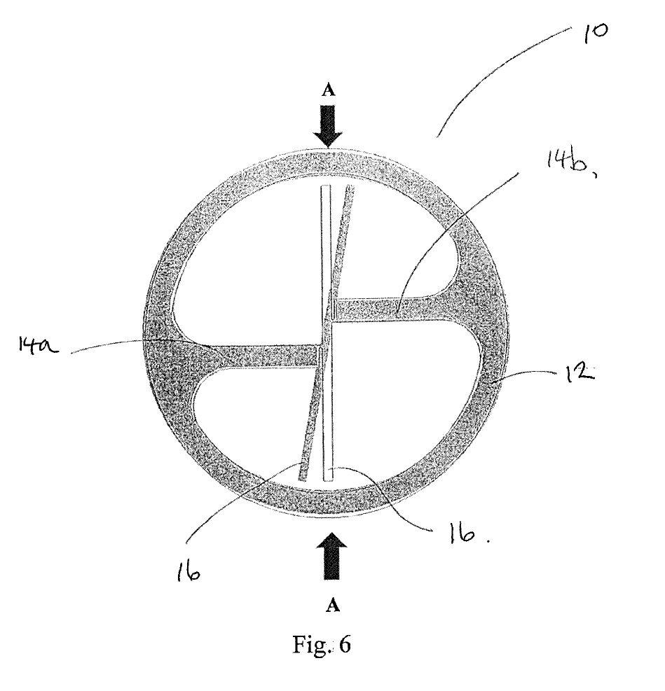

FIG. 6 is a top elevation view of the device of FIG. 1 for detecting deformation of a hollow component in a deformed state following the application of compressive forces to the device in the direction of the arrows A.

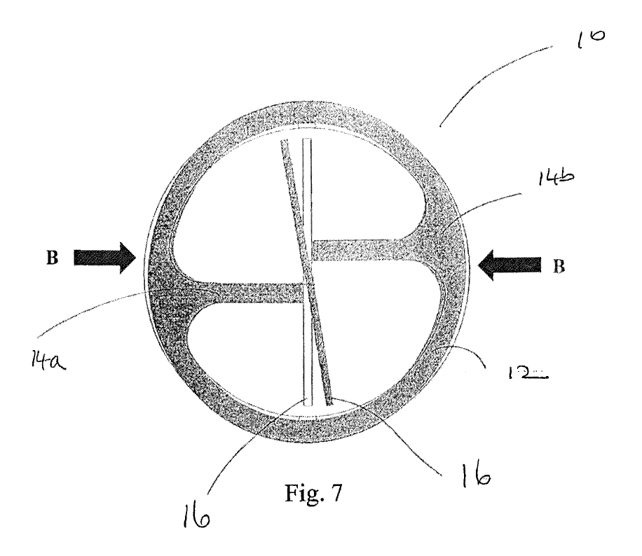

FIG. 7 is a top elevation view of the device for detecting deformation of a hollow component in a deformed state following the application of compressive forces to the device in the direction of the arrows B.

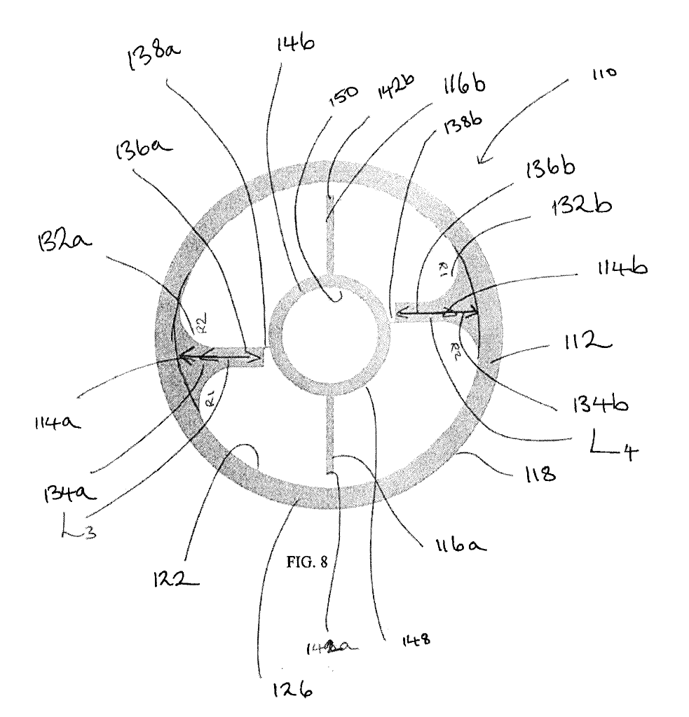

FIG. 8 is a top elevation view of a second construction of a device for detecting deformation of a hollow component in an un-deformed state.

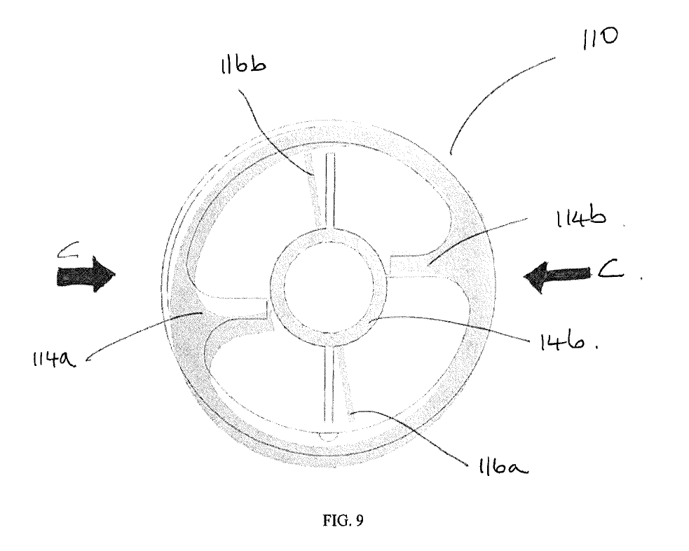

FIG. 9 is a top elevation view of the device of FIG. 8 for detecting deformation of a hollow component in a deformed state following the application of compressive forces to the device in the direction of the arrows C.

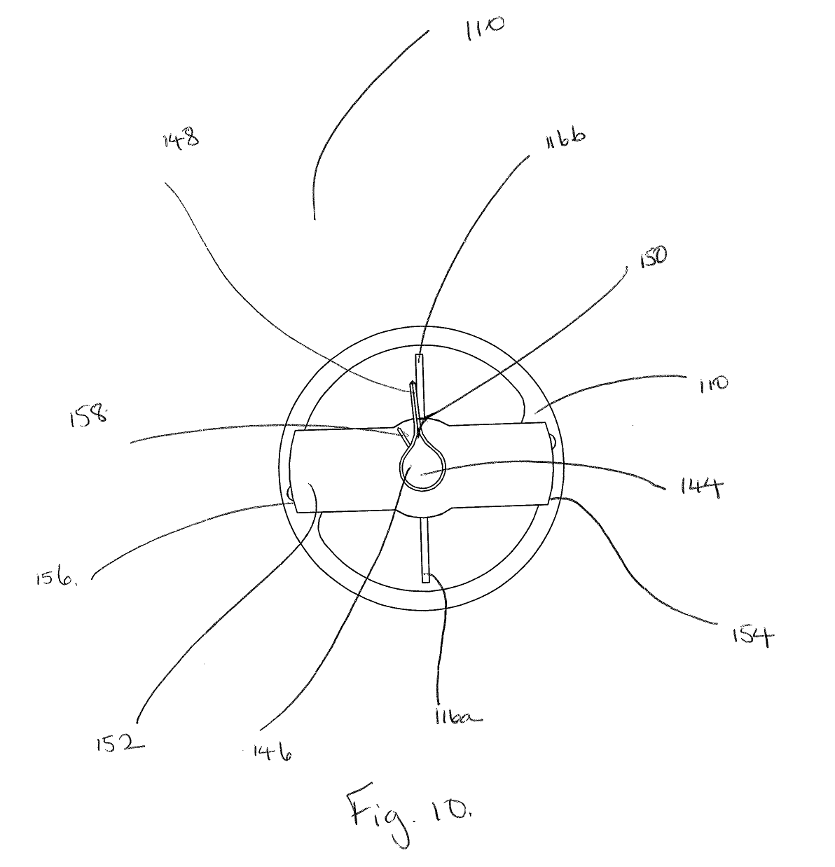

FIG. 10 is a top elevation view of the second construction of the device which includes a marker for indicating the largest angular deflection of the elongate indicator.

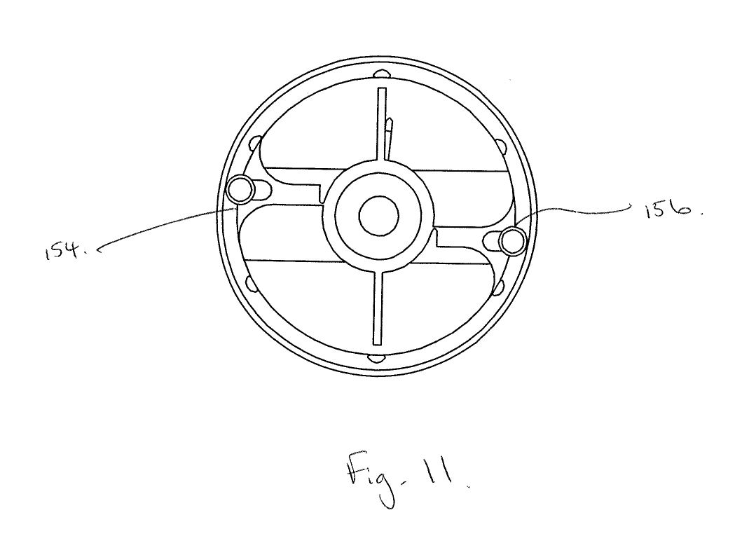

FIG. 11 is a bottom elevation view of the device of FIG. 10.

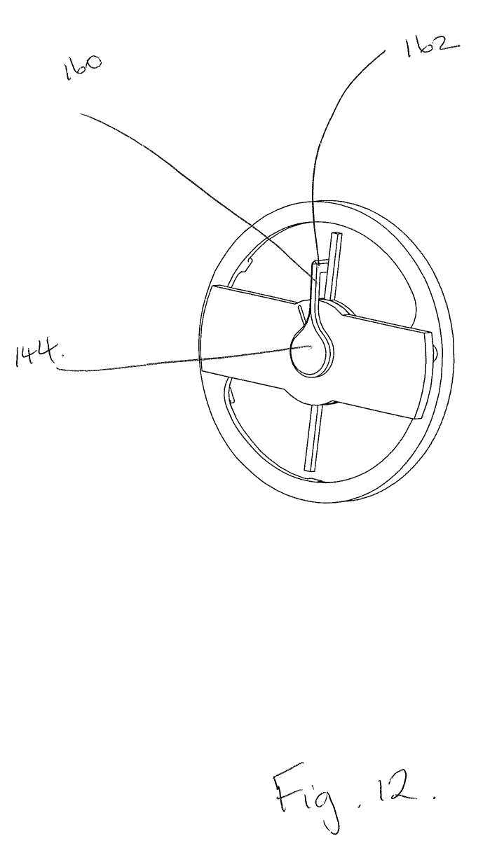

FIG. 12 is side elevation view of the device of FIG. 10

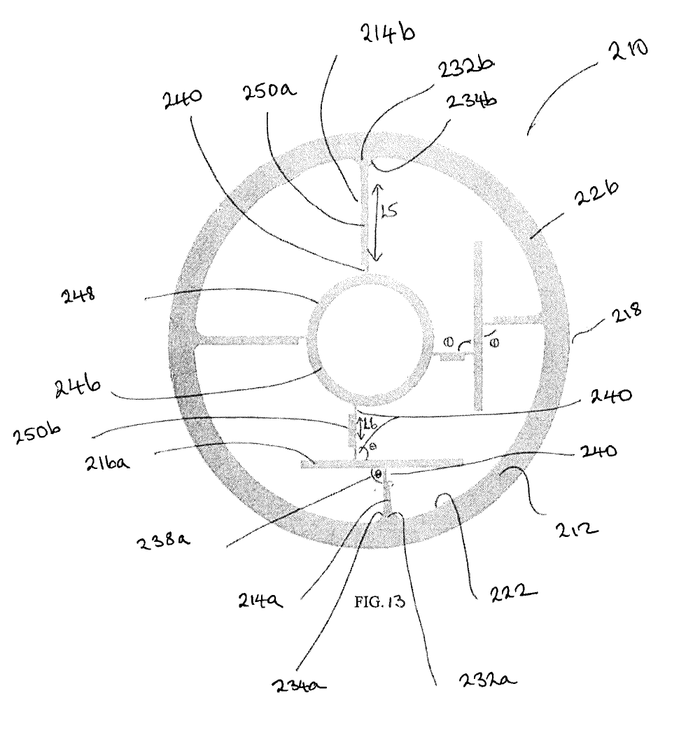

FIG. 13 is a top elevation view of a third construction of a device for detecting deformation of a hollow component in an un-deformed state. The device has two pairs of arms. Details of the first pair of arms are described.

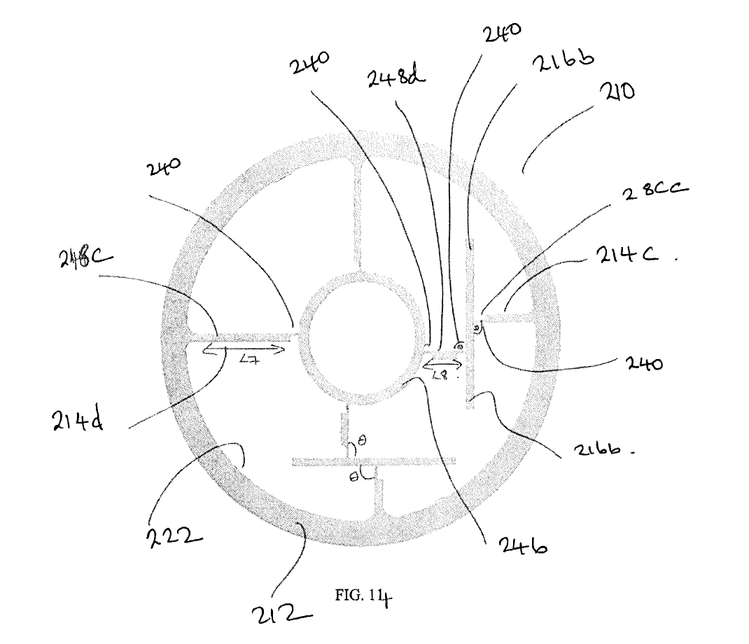

FIG. 14 is a top elevation view of a third construction of a device as shown in FIG. 10. Details of the second pair of arms are described.

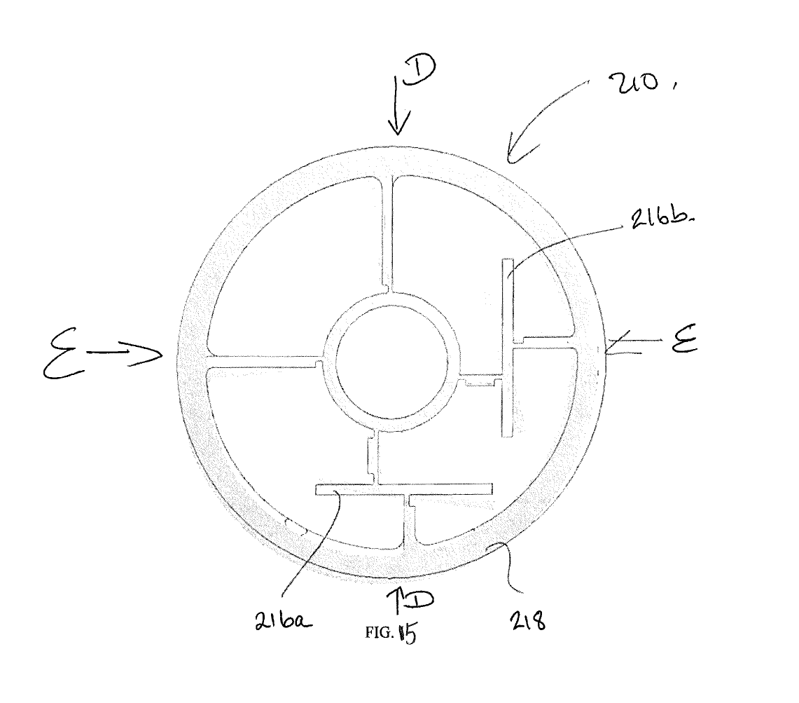

FIG. 15 is a top elevation view of the device of FIG. 10 for detecting deformation of a hollow component in a deformed state following the application of compressive forces to the device in the direction of the arrows D and E.

Referring now to FIGS. 1 to 7, there is shown a device 10 for detecting deformation of a hollow component. The device includes a frame 12, a pair of arms 14a, 14b and a needle 16. The device is shown as being provided as one piece. This construction can be achieved by manufacturing the parts of the device by a moulding technique, such as injection moulding.

The frame 12 of the depicted construction has an annular structure. The frame 12 can have other shapes, but it is advantageous that the shape of the frame's outer periphery 18 complements the internal shape of the rim of the hollow component into which it will be fitted. This ensures that the device 10 fits snugly within the rim of the hollow component and is therefore capable of detecting small amounts of deformation. The outer periphery 18 of the frame 12 is defined by an outer wall 20. An inner periphery 22 of the frame 12 is defined by an inner wall 24.

The frame 12 has a first side 26 and an opposite second side 28. The inner and outer walls extend between the edges of the first and second sides. In certain constructions of the device, the first side 26 and the second side 28 of the device are identical. As a result, there is not a requirement that the device 10 is inserted into the hollow component in any specific orientation. By this, it is meant that the device can be inserted into the hollow component with either the first side 26 or the second side 28 facing upwards (i.e., visible to the user).

In other constructions, for example as illustrated in FIG. 2, an anti-rotation feature 30 is provided on at least one of the first side 26 or the second side 28. In the construction illustrated in FIG. 2, the anti-rotation feature 30 takes the form of a plurality of protrusions that are distributed on the second side 28. Each protrusion is configured to mate with a complementary feature (for example, a recess) formed within the inner surface of the hollow component. The anti-rotation feature 30 prevents or significantly limits the ability of the device 10 being rotated in the hollow component during use. This is advantageous because it enables the user to accurately determine the specific location of any deformation.

However, in some circumstances it may be advantageous for the user to be able to rotate the device within the hollow component in order to locate the direction of greatest deflection of the needle. This informs the user of the region of greatest deformation of the hollow component. In such circumstances, the user can either insert a construction of the device that does not have any anti-rotation features into the hollow component, or if anti-rotation features are provided on one side of the device, orientate the device so that the side provided with the anti-rotation features is facing upwards (i.e., visible to the user).

Each arm 14a, 14b has a frame-connecting end 32a, 32b. In the depicted construction of the device, the frame-connecting end 32a, 32b of the each arm 14a, 14b is connected to the inner periphery 22 of the frame 12 via a flared neck portion 34a, 34b. Each flared neck portion 34a, 34b has a curved outer profile extending between the inner periphery 22 of the frame and the parallel longitudinal portions 36a, 36b of the arm 14a, 14b. The flared neck portion 34a, 34b has a first radius of curvature (R₁) connecting the inner periphery 22 of the frame 12 and a first longitudinal portion 36a of an arm 14a, 14b, and a second radius of curvature (R₂) connecting the inner periphery 22 of the frame 12 and a second longitudinal portion 36b of arm 14a, 14b. In the depicted construction, the first radius of curvature (R₁) is greater than the second radius of curvature (R₂). The flared neck portion 32a, 32b provides rigidity to the arms.

Each arm 14a, 14b also has a needle-connecting end 38a, 38b. In the depicted construction the needle-connecting end 38a, 38b of each arm 14a, 14b is connected to the needle 16 at an angle (“θ”—see FIG. 5) of about 90° prior to any deformation of the device. It is however envisaged that the angle (θ) between one or each of the arms 14a, 14b, and the portion of the needle that extends between the arms, can be at least about 60°, or at least about 70°, or at least about 80°. Generally, the angle will be not more than about 120°, or not more than about 110°, or not more than about 100°. FIG. 5 is an enlarged view of the area “A” shown in FIG. 1, and details the design of the hinge 40 that allows the angle(s) between the arms 14a, 14b and the needle 16 to change when the device 10 is deformed. The hinge 40 is provided by a portion of the arms 14a, 14b that is thinner compared to the adjacent portion of the arm 14a, 14b. The material of the illustrated hinge has a thickness that is approximately 1/7^{th} the thickness of the adjacent portion of each arm 14a, 14b. The thinned hinge portion of each arm 14a, 14b is rounded when viewed from one side so that there is no sharp change in wall thickness at the point at which the hinge 40 will flex when the device is deformed.

Each arm 14a, 14b has a longitudinal axis (“L₁, L₂”—see FIG. 2). The length of each of the arms is measured along the longitudinal axis. The longitudinal axis (L₁, L₂) extends though the flared neck portion 34a, 34b and intersects a line representing the continuation of the inner periphery 22 of the frame 12. In the construction shown in FIGS. 1 to 7, the length of the first arm 14a is equal in length to the second arm 14b. The ratio of the length of each of the arms 14a, 14b to the distance between the arms measured along the needle is about 0.15. This ratio could be varied in order to maximise the angular displacement relative to the deformation.

As illustrated in FIG. 1 and FIG. 2, the first arm 14a is approximately parallel with the second arm 14b in a non-deformed state. It is envisaged that in some constructions the first and second arms are not approximately parallel in a non-deformed state.

The needle 16 extends diametrically across the frame 12. The needle has free ends 42a, 42b. In the depicted construction each of the ends 42a, 42b of the needle 16 is blunt. Optionally, at least one of the ends 42a, 42b of the needle 16 can be pointed.

Referring now to FIGS. 6 and 7, the device 10 is shown in both a non-deformed state (without shading) and a deformed state (with shading). Any deformation of the hollow component (not shown) is visualised as a deflection of the needle 16. This is advantageous because small amounts of deformation of the hollow component, which are difficult to detect with the human eye but which can be clinically critical, are detectable. FIGS. 6 and 7 illustrate the deformation of the outer periphery 18 of the frame 12. Deformation of the frame occurs when the outer periphery 22 of the device is placed against a deformed region of a hollow component. This deformed region will generate compressive forces that act upon the outer periphery 18 of the frame 12. Examples of the direction of these compressive forces is illustrated by arrows “A” and “B”. The deformation of the outer periphery 18 of the frame 12 causes movement of the first and second arms 14a, 14b, which leads to the needle 16 being angularly displaced from its original position. Although not illustrated, a scale can be provided on the frame which enables the user to quantify the amount of deflection of the needle. This will allow the user to determine if a certain amount of deformation of the hollow component has been attained or exceeded.

Referring now to FIGS. 8 to 12, there is shown a second construction of a device 110 for detecting deformation of a hollow component. The device includes a frame 112, a pair of arms 114a, 114b and a needle 116. The device is shown as being provided as one piece. This construction can be achieved by manufacturing the parts of the device by a moulding technique, such as injection moulding.

The frame 112 of the depicted construction has an annular structure. The frame 112 can have other shapes, but it is advantageous that the shape of the frame's outer periphery 118 complements the internal shape of the rim of the hollow component into which it will be fitted. This ensures that the device 110 fits snugly within the rim of the hollow component and is therefore capable of detecting small amounts of deformation. The outer periphery 118 of the frame 112 is defined by an outer wall (not shown). An inner periphery 122 of the frame 112 is defined by an inner wall (not shown).

The frame 112 has a first side 126 and an opposite second side (not shown). The inner and outer walls extend between the edges of the first and second sides. In certain constructions of the device, the first side 126 and the second side of the device are identical. As a result, there is not a requirement that the device 110 is inserted into the hollow component in any specific orientation. By this, it is meant that the device can be inserted into the hollow component with either the first side 126 or the second side facing upwards (i.e., visible to the user). In other constructions, an anti-rotation feature (not shown) is provided on at least one of the first side 126 or the second side. The anti-rotation feature can take the form of a plurality of protrusions that are distributed on the second side. Each protrusion is configured to mate with a complementary feature (for example, a recess) formed within the inner surface of the hollow component. The anti-rotation feature prevents or significantly limits the ability of the device 110 being rotated in the hollow component during use. This is advantageous because it enables the user to accurately determine the specific location of any deformation.

Each arm 114a, 114b has a frame-connecting end 132a, 132b. In the depicted construction of the device, the frame-connecting end 132a, 132b of each arm 114a, 114b is connected to the inner periphery 122 of the frame 112 via a flared neck portion 134a, 134b. Each flared neck portion 134a, 134b has a curved outer profile extending between the inner periphery 122 of the frame and the parallel longitudinal portions 136a, 136b of the arm 114a, 114b. The flared neck portion 134a, 134b has a first radius of curvature (R₁) connecting the inner periphery 122 of the frame 112 and a first longitudinal portion 136a of an arm 114a, 114b, and a second radius of curvature (R₂) connecting the inner periphery 122 of the frame 112 and a second longitudinal portion 136b of arm 114a, 114b. In the depicted construction, the first radius of curvature (R₁) is greater than the second radius of curvature (R₂). The flared neck portion 132a, 132b provides rigidity to the arms.

The needle 116a extends diametrically across the frame 112 and includes a first portion 116a and a second portion 116b that are separated by an inner frame 146. Each needle portion 116a, 116b extends from an opposing side of the external periphery 148 of the inner frame 146. The inner frame 146 of the depicted construction has an annular structure. The inner frame 146 can have other shapes, but it is advantageous that the shape of the inner frame's inner periphery 150 complements the external shape of the shaft of an acetabular cup inserter (not shown). The inner frame 146 is preferably constructed from a material that has a sufficient rigidity to ensure that the frame is able to retain its annular shape during insertion into the device. This ensures that the inner frame 146 does not cause an obstruction to the insertion of the cup inserter. The needle 116 has free ends 142a, 142b. In the depicted construction each of the ends 142a, 142b of the needle 116 is blunt. Optionally, the at least one of the ends 142a, 142b of the needle 116 can be pointed.

Each arm 114a, 114b also has a needle-connecting end 138a, 138b. In the depicted construction the needle-connecting end 138a, 138b of each arm 114a, 114b is indirectly connected to the needle 116 via connection to the external periphery 148 of the inner frame 146. Each arm 114a, 114b is connected at an angle (“θ”—see FIG. 8) of about 90° prior to any deformation of the device. It is however envisaged that the angle (θ) between one or each of the arms 114a, 114b, and the portion of the external periphery 148 of the inner frame 146 to which the arms are connected, can be at least about 60°, or at least about 70°, or at least about 80°. Generally, the angle will be not more than about 120°, or not more than about 110°, or not more than about 100°. A hinge 140 allows the angle(s) between the arms 114a, 114b and the indicator 116 to change when the device 110 is deformed. The hinge 140 is provided by a portion of the arms 114a, 114b that is thinner compared to the adjacent portion of the arm 114a, 114b. Advantageously, the thinned hinge portion of each arm 114a, 114b is rounded when viewed from one side so that there is no sharp change in wall thickness at the point at which the hinge 140 will flex when the device is formed.

Each arm 114a, 114b has a longitudinal axis (“L₃, L₄”—see FIG. 8). The length of each of the arms is measured along the longitudinal axis. The longitudinal axis (L₃, L₄) extends though the flared neck portion 134a, 134b and intersects a line representing the continuation of the inner periphery 122 of the frame 112. In the construction shown in FIGS. 8 and 9, the length of the first arm 114a is equal in length to the second min 114b. The ratio of the length of each of the arms 114a, 114b to the distance between the arms measured along the needle is about 0.3.

As shown in FIGS. 8 and 9, the first arm 114a is approximately parallel with the second arm 114b in a non-deformed state.

Referring now to FIG. 9, the device 110 is shown in both a non-deformed state (without shading) and a deformed state (with shading). Any deformation of the hollow component (not shown) is visualised as a deflection of the first 116a and second portions 116b of the needle 116. This is advantageous because small amounts of deformation of the hollow component, which are difficult to detect with the human eye but which can be clinically critical, are detectable. Deformation of the frame occurs when the outer periphery 122 of the device is placed against a deformed region of a hollow component. This deformed region will generate compressive forces that act upon the outer periphery 118 of the frame 112. Examples of the direction of these compressive forces is illustrated by arrows “C”. The deformation of the outer periphery 118 of the frame 112 causes movement of the first and second arms 114a, 114b. As the arms 114a, 114b move away from being parallel with each other, the needle 116 is angularly displaced from its original position. Although not illustrated, a scale can be provided on the frame which enables the user to quantify the amount of deflection of the needle. This will allow the user to determine if a certain amount of deformation of the hollow component has been attained or exceeded.

Referring to FIG. 10, the device 110 includes a marker 144. The marker can be used to indicate the largest deflection of the needle portion 116b which has occurred when the device is placed within the hollow component in two or more orientations. In the illustrated construction, the marker 144 is a pivotable element. The needle portion 116b and the marker 144 are each rotatably mounted for rotation about a common rotation axis.

The indicator has a generally tear-dropped shaped body 146 with an L-shaped element 148 extending from the narrowest point 150. A first arm 160 of the L-shaped element 144 is planar to the tear-dropped shaped body 146. The second arm 162 of the L-shaped element 144 extends downwardly towards the needle portion 116b.

A plate 152 extends from a third side 154 of the frame to an opposing fourth side 156 of the frame. The plate may be permanently secured to the frame, for example by rivets. Optionally, the plate is removably connectable to the frame, for example by a snap-fit connection. This allows the user to choose whether or not to use the marker. In some circumstances the user may not want the view of the interior of the hollow component to be obscured by the frame.

The indicator is connected to an upper side of plate 152.

The plate includes an indicium 158, illustrated here in the form of line, which represents the acceptable tolerance of the hollow component to deformation. If the marker deflects past this point, the user is aware that the deformation of the hollow component is outside of acceptable tolerance limits. The indicium can provide a qualitative indication of the amount of angular deflection of the elongate indicator, such as a “pass” or “fail”. In other constructions, indicia may be provided in the form of a scale, which can provide a quantitative indication of the amount of angular deflection of the needle portion 116b.

When the device is placed into the hollow component, a side surface of the second arm portion 162 of L-shaped element 148 generally abuts a side surface of the needle portion 116b. When the needle portion 116b is angularly deflected as a result of the device detecting a deformation in the inner surface of the hollow component, the second arm portion 162 is pushed anti-clockwise by the needle portion 116b. This causes the marker 144 to be moved from its first (original) position to a second position. Advantageously, the marker is configured so that it does not automatically revert to its original position, but requires user input. This may be achieved, for example, as a result of frictional resistance or a ratchet mechanism.

When a user removes the device from the hollow component the needle portion 116b will be restored to its original position but the marker 144 will remain in its second position. It can only be returned to its first position by the user.

The device may be rotated within the hollow component by the user in order to measure the maximum deflection of the needle portion 116b and thus provide the user with an indication of the maximum deformation in all directions of the hollow component.

If the second angular deflection of needle portion 116b is greater than the first angular deflection of needle portion 116b, the marker 144 is moved in an anticlockwise direction from the second position to the third position. If the second angular deflection of needle portion 116b is equal to or less than the first angular deflection of needle portion 116b, the marker 144 will remain in the second position.

The fact that the marker does not automatically revert to its original position enables the user to assess the deformation of the hollow component at various points about its inner surface by either rotating the device within the hollow component or repeatedly removing and re-positioning the device within the hollow component. The marker indicates to the user the maximum deflection of the needle portion 116b that has occurred during the assessment. This provides the user with an indication of the maximum deformation of the hollow component.

In the construction shown, if the L-shaped element 148 has been deflected past the indicium 158, the user knows that the deformation of the hollow component is outside of acceptable tolerance limits.

Referring now to FIGS. 13 to 15, there is shown a third construction of a device 210 for detecting deformation of a hollow component. The device is shown as being provided as one piece. This construction can be achieved by manufacturing the parts of the device by a moulding technique, such as injection moulding.

The device includes a frame 212, which is shown as having an annular structure. The frame 212 can have other shapes, but it is advantageous that the shape of the frame's outer periphery 218 complements the internal shape of the rim of the hollow component into which it will be fitted. This ensures that the device 210 fits snugly within the rim of the hollow component and is therefore capable of detecting small amounts of deformation. The outer periphery 218 of the frame 212 is defined by an outer wall (not shown). An inner periphery 222 of the frame 212 is defined by an inner wall (not shown).

The frame 212 has a first side 226 and an opposite second side (not shown). The inner and outer walls extend between the edges of the first and second sides. In certain constructions of the device, the first side 226 and the second side of the device are identical. As a result, there is not a requirement that the device 210 is inserted into the hollow component in any specific orientation. By this, it is meant that the device can be inserted into the hollow component with either the first side 226 or the second side facing upwards (i.e., visible to the user). In other constructions, an anti-rotation feature (not shown) is provided on at least one of the first side 226 or the second side. The anti-rotation feature can take the form of a plurality of protrusions that are distributed on the second side. Each protrusion is configured to mate with a complementary feature (for example, a recess) formed within the inner surface of the hollow component. The anti-rotation feature prevents or significantly limits the ability of the device 210 being rotated in the hollow component during use. This is advantageous because it enables the user to accurately determine the specific location of any deformation.

The device is provided with a first pair of arms (214a, 214b) and a second pair of arms (214c, 214d). The provision of two pairs of arms enables the deformation of the hollow component to be detected in two different axis without requiring rotation of the device.

As shown in FIG. 13, each arm of the first pair of arms 214a, 214b has a frame-connecting end 232a, 232b. In the depicted construction of the device, the frame-connecting end 232a, 232b is connected to the inner periphery 222 of the frame 212 via a flared neck portion 234a, 234b. Each flared neck portion 234a, 234b has a curved outer profile extending between the inner periphery 222 of the frame and the parallel longitudinal portions of the each arm 214a, 214b.

The first arm 214a of the first pair of arms 214a, 214b has a needle-connecting end 238a that connects the arm to a needle 216a via a hinge portion 240.

The second arm 214b of the first pair of arms 214a, 214b includes a first arm portion 250a that extends inwardly from the inner periphery 222 of the frame 212 and connects to the external periphery 248 of an inner frame 246 via a hinge 240. The second arm 214b also includes a second arm portion 250b that is connected to the external periphery 248 of the inner frame 246 via a hinge 240, and extends outwardly from the inner frame 246 to connect, via a hinge 240, with the needle 216a. The second arm portion 250b of the second arm 214b is connected to the needle 216a at a spaced apart point along the length of the needle 216a from which the first arm 214a is connected to the needle 216a.

The hinges 240 can be formed by a thinner region of material. Advantageously, the thinned hinge region is rounded when viewed from one side so that there is no sharp change in wall thickness at the point at which the hinge 240 will flex when the device is deformed.

The first arm portion 250a and second arm portion 250b each have a longitudinal axis (“L₅, L₆”). In the embodiment shown in FIG. 10 the first arm portion 250a and second arm portion 250b are connected to the inner frame 246 such that L₅, L₆ are off-set from each other. Other constructions are envisaged, for example in which the longitudinal axis L₅ of the first portion 250a and the longitudinal axis L₆ of the second portion 250b are connected to the inner frame 246 such that they are aligned with each other.

Each arm 214a, 214b is connected to needle 216a at an angle (“θ”) of about 90° prior to any deformation of the device. It is however envisaged that the angle (θ) between one or each of the arms 214a, 214b, and the needle 216a, can be at least about 60°, or at least about 70°, or at least about 80°. Generally, the angle will be not more than about 120°, or not more than about 110°, or not more than about 100°.

The third construction of the device as shown in FIG. 13 is also provided with a second pair of arms. The details of the second pair of arm is provided with reference to FIG. 11. The second pair of arms is identical to the first pair of arms. In the third construction, the angle (“θ”) between the needle of the first pair of arms and the needle of the second pair of aims is approximately 90°. This enables deformation of the device in two orthogonal axes be detected.

The first arm 214c of the second pair of arms 214c, 214d has a needle-connecting end 238c that connects the arm to a needle 216b via a hinge portion 240.

The second arm 214d of the second pair of arms 214c, 214d includes a first arm portion 248c that extends inwardly from the inner periphery 222 of the frame 212 and connects to the inner frame 246 via a hinge 240. The second arm 214d also includes a second arm portion 248d that is connected to the inner frame 246 via a hinge 240, and extends outwardly from the inner frame 246 to connect, via a hinge 240, with the needle 216b. The second arm portion 248d of the second arm 214d is connected to the needle 216b at a spaced apart point along the length of the needle 216b from which the first arm 214c is connected to the needle 216b.

The hinges 240 can be formed by a thinner region of material. Advantageously, the thinned hinge region is rounded when viewed from one side so that there is no sharp change in wall thickness at the point at which the hinge 240 will flex when the device is deformed.

The first arm portion 248c and second arm portion 248d each have a longitudinal axis (“L₇, L₈”). In the construction shown in FIG. 14 the first arm portion 248c and second arm portion 248d are connected to the inner frame 246 such that L₇, L₈ are off-set from each other. Other constructions are envisaged, for example in which the longitudinal axis L₇ of the first portion 248a and the longitudinal axis L₇ of the second portion 248b are connected to the inner frame 212 such that they are aligned with each other.

Each arm 214c, 214d is connected to needle 216b at an angle (“θ”) of about 90° prior to any deformation of the device. It is however envisaged that the angle (θ) between one or each of the arms 214c, 214d, and the portion of the external periphery 148 of the inner frame 246 to which the arms are connected, can be at least about 60°, or at least about 70°, or at least about 80°. Generally, the angle will be not more than about 120°, or not more than about 110°, or not more than about 100°.

The inner frame 246 of the construction shown in FIGS. 13 to 15 has an annular structure. The inner frame 246 can have other shapes, but it is advantageous that the shape of the inner frame's inner periphery 250 complements the external shape of the shaft of an acetabular cup inserter (not shown). The inner frame 246 is preferably constructed from a material that has a sufficient rigidity for the frame to retain its shape during use. This ensures that the inner frame does not cause an obstruction to the insertion of the cup inserter.

Referring now to FIG. 15, the device 210 is shown in both a non-deformed state (without shading) and a deformed state (with shading). Any deformation of the hollow component (not shown) is visualised as a deflection the first needle 216a and/or the second needle 216b. This is advantageous because small amounts of deformation of the hollow component, which are difficult to detect with the human eye but which can be clinically critical, are detectable. Deformation of the frame occurs when the outer periphery 218 of the device is placed against a deformed region of a hollow component. This deformed region will generate compressive forces that act upon the outer periphery 218 of the frame 212. Examples of the direction of these compressive forces are illustrated by arrows “D” and “E”.

The deformation of the outer periphery 218 of the frame 212 by a compressive force in the direction of arrow D results in movement of the first arm 214a and the second 214b arm of the first pair of arms. As the arms 214a, 214b move away from being parallel with each other the needle 116a is angularly displaced from its original position. Although not illustrated, a scale can be provided on the frame which enables the user to quantify the amount of deflection of the needle. This will allow the user to determine if a certain amount of deformation of the hollow component has been attained or exceeded.

The deformation of the outer periphery 218 of the frame 212 by a compressive force in the direction of arrow E results in movement of the first arm 214c and the second arm 214d of the second pair of arms. As the arms 214c, 214d move away from being parallel with each other, the needle 116b is angularly displaced from its original position. Although not illustrated, a scale can be provided on the frame which enables the user to quantify the amount of deflection of the needle. This will allow the user to determine if a certain amount of deformation of the hollow component has been attained or exceeded.

1. A device for fitting into a hollow component to provide an indication of deformation of the hollow component, the device comprising:
a frame, and
an elongate indicator suspendedly connected to the frame, the elongate indicator being arranged to deflect as a result of deformation of the frame.

2. The device of claim 1, further comprising:
a pair of arms whereas a first arm extends inwardly from a first point on a first side of the frame, and a second arm extends inwardly from a second point on a second side of the frame, the second side being opposite to the first side,
whereas the elongate indicator has a longitudinal axis to which the first and second arms are connected at spaced apart points along the axis to suspendedly connect the elongate indicator to the frame, and
whereas a change in the distance between the first and second points on opposite sides of the frame caused by deformation of the frame results in angular deflection of the indicator.

3. The device of claim 2, whereas the indicator is a needle, the first and second arms being connected to the needle at spaced apart points along the length of the needle.

4. The device of claim 1, further comprising a second elongate indicator suspendedly connected to the frame, the elongate indicator being arranged to deflect as a result of deformation of the frame.

5. The device of claim 4, whereas the device further comprises:
a second pair of arms whereas a third arm extends inwardly from a third point on a third side of the frame, and a fourth arm extends inwardly from a fourth point on a fourth side of the frame, the fourth side being opposite to the third side,
whereas the second elongate indicator has a longitudinal axis to which the third and fourth arms are connected at spaced apart points along the axis to suspendedly connect the second elongate indicator to the frame, and
whereas a change in the distance between the third and fourth points on opposite sides of the frame caused by deformation of the frame results in angular deflection of the indicator.

6. The device of claim 5, whereas one of the third and fourth arms comprises a first arm portion and a second arm portion, the second arm portion being separated from the first arm portion by an inner frame.

7. The device of claim 2, whereas one of the first and second arms comprises a first arm portion and a second arm portion, the first arm portion being separated from the second arm portion by an inner frame.

8. The device of claim 1, whereas the indicator comprises a first needle portion, a second needle portion and an inner frame from which the first needle portion and second needle portion extend outwardly from opposite points.

9. The device of claim 2, whereas each of the arms of the pair of arms are approximately parallel with each other when there is no deformation of the frame.

10. The device of claim 2, whereas the angle between each of the arms of the pair of arms and the needle is approximately 90° when there is no deformation of the device.

11. The device of claim 2, whereas a hinge is provided between each of the arms of the pair of arms and the needle.

12. The device of claim 11, whereas each of the hinges between each of the arms of the pair of arms and the needle is provided by a locally thinned portion of the arm.

13. The device of claim 11, whereas each of the hinges is provided at the end of its respective arm at which the arm is connected to the needle.

14. The device of claim 1, whereas the frame is continuous around the periphery of the device.

15. The device of claim 14, whereas the frame is circular.

16. The device of claim 14, which includes indicia on the frame for indicating the amount of deflection of the indicator.

17. The device of claim 1 , whereas the device further includes a marker rotatably mounted on the frame and arranged for deflection by the elongate indicator to thereby mark a largest angular deflection of the elongate indicator from a series of angular deflections of the elongate indicator when the device is placed within the hollow component in two or more orientations.

18. The device of claim 17, whereas the marker is angularly deflected from a first position to a second position in response to a first angular deflection of the elongate indicator and not automatically returned to the first position and angularly deflected from the second position to a third position in response to a second angular deflection of the elongate indicator when the second angular deflection is greater than the first angular deflection.

19. The device of claim 17, whereas the elongate indicator and the marker are each rotatably mounted for rotation about a common rotation axis.

20. The device of claim 17, whereas the device further includes a plate extending between the first and second points on the frame, and whereas the marker is connected to the plate.

21. The device of claim 20, whereas the plate includes indicia for indicating the angular deflection of the marker.

22. The device of claim 1, whereas the frame and the indicator are moulded together in one piece.

23. A kit comprising:
a device having a frame, and an elongate indicator suspendedly connected to the frame, the elongate indicator being arranged to deflect as a result of deformation of the frame; and
a hollow component, whereas the hollow component has a rim and the device fits snugly within the rim of the hollow component, with the frame in contact with the rim.

24. The kit of claim 23, whereas the device is preassembled within the hollow component.

25. A kit of claim 23, whereas the hollow component is an orthopaedic component.

26. The kit of claim 25, whereas the orthopaedic component is for positioning in a cavity in a patient's acetabulum in a surgical procedure to implant a hip joint prosthesis.

27. A kit comprising first and second devices, each of the first and second devices having:
a frame;
an elongate indicator suspendedly connected to the frame, the elongate indicator being arranged to deflect as a result of deformation of the frame; and
a pair of arms whereas a first arm extends inwardly from a first point on a first side of the frame, and a second arm extends inwardly from a second point on a second side of the frame, the second side being opposite to the first side;
whereas the elongate indicator has a longitudinal axis to which the first and second arms are connected at spaced apart points along the axis to suspendedly connect the elongate indicator to the frame;
whereas a change in the distance between the first and second points on opposite sides of the frame caused by deformation of the frame results in angular deflection of the indicator; and
whereas the length of the first pair of arms of the first device are different from the length of the first pair of arms of the second device.

28. A method of detecting deformation of a hollow component, the method comprising the step of detecting angular deflection of an elongate indicator within a device fitted within the hollow component, the device comprising:
a flexible frame, and
an elongate indicator suspendedly connected to the frame, the elongate indicator being arranged to deflect as a result of deformation of the frame.

29. The method of claim 28, whereas the deformation of the frame is caused during insertion of the hollow component into a cavity.

30. The method of claim 28, whereas the deformation of the frame is caused during manufacture, transportation or storage of the hollow component.

31. The method of claim 28, whereas the hollow component is a component of an orthopaedic joint prosthesis.

32. The method of claim 31, whereas the orthopaedic component is an acetabular cup component of a hip joint prosthesis.

33. The method of claim 32, whereas the device is fitted within the acetabular cup prior to implantation of the acetabular cup into a patient's acetabulum.

34. The method of claim 28, whereas the device further includes a marker rotatably mounted on the frame and arranged for deflection by the elongate indicator to thereby mark a largest angular deflection of the elongate indicator from a series of angular deflections of the elongate indicator when the device is placed within the hollow component in two or more orientations and whereas the method further comprises the step of detecting the largest angular deflection of the elongate indicator when the device is placed within the hollow component in two or more orientations.
